(19) United States
(12) Patent Application Publication
(10) Pub. No.: US 2015/0045728 A1
(43) Pub. Date: Feb. 12, 2015

(54) DEVICE FOR TREATING CHRONIC TOTAL OCCLUSION

(71) Applicant: Richard R. Heuser, Phoenix, AZ (US)

(72) Inventor: Richard R. Heuser, Phoenix, AZ (US)

(21) Appl. No.: 14/509,551

(22) Filed: Oct. 8, 2014

Related U.S. Application Data
(63) Continuation of application No. 11/433,198, filed on May 11, 2006, now abandoned.
Application No. 11/433,198 is a continuation-in-part of application No. 10/272,317, filed on Oct. 15, 2002, now Pat. No. 7,179,250.
Application No. 10/272,317 is a continuation of application No. 09/705,963, filed on Nov. 3, 2000, now Pat. No. 6,464,681.
Application No. 09/705,963 is a continuation of application No. 09/397,806, filed on Sep. 17, 1999, now Pat. No. 6,159,197.

(51) Int. Cl. A61M 25/10; A61B 18/14; A61B 18/20; A61M 5/158

(52) U.S. Cl. CPC A61M 25/10; A61M 5/158; A61B 18/1492; A61B 18/201; A61M 2025/105; A61M 2025/1052; A61M 2025/1063; A61M 2205/054; A61B 2017/00261
USPC 604/103.11

(57) ABSTRACT

A catheterization system for treatment of a condition within a blood vessel is provided with a catheter, a balloon immediately adjacent to the distal end of the catheter, an inflation device for expanding the balloon, and a fluid injection device for injecting a treatment fluid through the central lumen of the catheter and out of the aperture at the distal end of the catheter into the treatment area. The system may include an RF wire, a hollow needle wire, a dissection tool, or a laser wire insertable through the central lumen of the catheter and selectively operable adjacent the proximal end of the catheter to apply treatment within the blood vessel.

### RELATED APPLICATIONS

This application is a continuation of copending U.S. patent application Ser. No. 11/433,198, filed May 11, 2006, which is a continuation-in-part of U.S. patent application Ser. No. 10/272,317, filed Oct. 15, 2002, which issued on Feb. 20, 2007 as U.S. Pat. No. 7,179,250, and which was a continuation of U.S. patent application Ser. No. 091705,963, filed Nov. 3, 2000, which issued on Oct. 15, 2002 as U.S. Pat. No. 6,464,681 and which was a continuation of U.S. patent application Ser. No. 09/397,806, filed Sep. 17, 1999, which issued on Dec. 12, 2000 as U.S. Pat. No. 6,159,197, all of which are incorporated herein by reference.

### BACKGROUND AND SUMMARY

Disclosed herein is a device for treating a condition in a blood vessel, typically an artery, where plaque and/or other buildup or constriction has caused a complete or near-complete blocking or occlusion of the blood vessel. Typically the device is for treatment of such a condition of vascular occlusion that has existed for a period of at least a month and in some cases several months or years, although it may also be used in conditions of a shorter duration. The artery may be located anywhere in the body, typically in the legs, neck, or heart.

Treatment of heart disease has traditionally been a highly traumatic endeavor. For many years surgeons would be required to conduct major surgery to correct even relatively minor conditions. Such “open-heart” operations are highly traumatic for the patient and may therefore not be an option for those whose bodies cannot withstand such trauma. Open-heart operations are also expensive and may be risky. There is also a possibility of the patient contracting an infection during his or her extended stay in a medical care facility. For these reasons, some conditions may not merit treatment if open-heart surgery is required for their treatment.

The use of low-trauma surgery devices and techniques has increased the treatment and success rates for many conditions that are either too risky or too expensive to perform during open-heart surgery. The catheter is one such low-trauma device that has been especially successful in the treatment of cardiovascular and other conditions. A typical catheter is a flexible, hollow small-diameter tube that is threaded through a body system (such as the cardiovascular system) until it reaches a location that requires treatment. An advantage of a catheter is that only a small incision need be made to insert the catheter into the body. This significantly reduces the trauma experienced by the patient and dramatically reduces recovery time. Furthermore, depending on the procedure, only local anesthesia may be needed. This reduces the risk and cost of the procedure. Catheters have been successfully used in angioplasty procedures and in the delivery of stents and other medical devices into selected areas of the body.

One procedure that has met with limited success using low-trauma surgical techniques is the killing off or elimination of tissues such as the septum of the heart. If a tissue-killing substance such as alcohol is inserted into an artery leading to the septum, there is a risk that some of the alcohol may travel instead through arteries leading to other portions of the heart. This would damage other portions of the heart, and a heart attack may result. Known infusion techniques have not been able to reliably deliver alcohol to a desired tissue while preventing the alcohol from damaging other tissue.

Another aspect provides a method of introducing a tissue killing substance into a bodily fluid vessel. According to the method, a catheter is provided that has a blocking mechanism configured to selectively block and unblock the vessel. The catheter also has a delivery system that is configured to introduce the tissue-killing substance into the vessel. The vessel is substantially blocked upstream of a selected tissue using the blocking mechanism. The tissue-killing substance is introduced into the vessel through the delivery system, and the vessel is unblocked when the tissue-killing substance has substantially traveled toward the selected tissue.

The blocking mechanism may also be used to apply treatment to a vascular occlusion. The treatment may include infusion of liquid and/or the application of energy including radio-frequency, laser, or mechanical force. Vascular occlusions are more difficult to remove where the blockage includes a mineral component, typically a calcification. Such occlusions are difficult to reopen and, even if reopened, tend toward restenosis, i.e., a repeat of the occlusion. Treatment of the plaque and calcification with an appropriate substance will allow the reopening and reduce the chances of restenosis.

U.S. Pat. No. 6,290,689, which is incorporated herein by reference, discloses a catheter device for the treatment of calcified vascular occlusions.

### BRIEF DESCRIPTION OF THE DRAWINGS

FIG. 1 is a side elevational, partial cutaway view of a catheter with a balloon according to an embodiment of the disclosure.

FIG. 2 is a side elevational, cutaway view of a body fluid vessel with the catheter of FIG. 1 inserted therein on a guidewire.

FIG. 3 is a side elevational view of the catheter of FIG. 1 inserted in a blood vessel, the balloon inflated, and infusing a liquid into the vessel.

FIG. 4 is a side elevational, cutaway view of the catheter shown in FIG. 1 in a blood vessel with the balloon inflated adjacent an occluded portion of the vessel.

FIG. 5 is a side elevational, partially cutaway view of the catheter of FIG. 1 with a needle-tipped, hollow wire within an internal lumen of the catheter.

FIG. 6 is a side elevational, partially cutaway view of the catheter with the needle-tipped, hollow wire of FIG. 5 extending out of an aperture at the distal end of the catheter.

FIG. 7 is a side elevational, partially cutaway view of the catheter of FIG. 1 with a radio-frequency wire within an internal lumen of the catheter.

FIG. 8 is a side elevational, partially cutaway view of the catheter of FIG. 1 with a hollow wire within an internal lumen of the catheter, and a needle wire within the hollow wire.

FIG. 9 is a pictorial view of the proximal and distal ends of the needle-tipped, hollow wire of FIG. 5 showing the needle-tip at the distal end and a syringe coupled to the proximal end.

FIG. 10 is a side elevational, cutaway view of the radio-frequency wire treating an occluded portion of a blood vessel.

FIG. 11 is a side elevational, cutaway view of the needle-tipped, hollow wire treating an occluded portion of a blood vessel.

FIG. 12 is side elevational view of a catheter in accordance with an embodiment of the disclosure showing an ovally-shaped balloon.

FIG. 13 is a side elevational, cutaway view of the catheter with a wire having a dissection tool at its distal tip.

FIG. 14 is a side elevational, cutaway view of the dissection tool treating an occluded portion of the blood vessel.

### DETAILED DESCRIPTION

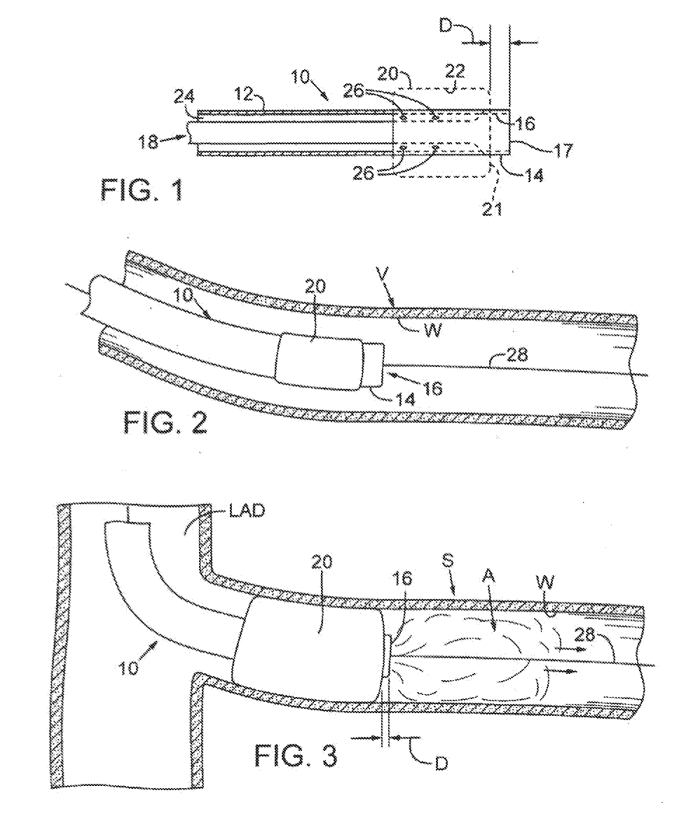

FIG. 1 depicts a first catheter 10 that may be used with the processes and procedures disclosed herein. First catheter 10 includes a flexible, generally cylindrical length of hollow tubing 12. The tubing preferably has an outside diameter of about 1-4 mm. A distal end 14 of the first catheter has an opening or aperture 16, which is defined by an annular rim or edge 17. A first passage, shown as a first lumen 18, runs the length of catheter 10 and communicates with aperture 16. First lumen 18 preferably has an inner diameter of about 0.018-0.038 inches. The first lumen permits fluids or colloids to be selectively introduced into a vessel, as will be described below.

A first flexible membrane, shown as a first balloon 20, is secured to tubing 12 adjacent distal end 14. First balloon 20 includes a distal end 21 that is preferably positioned at a distance D from rim 17 such that distal end 21 of balloon 20 is immediately adjacent aperture 16. As can be seen in FIG. 1, distance D is typically about one-half of the diameter of tubing 12, or about 0.5 mm to 2.0 mm. Alternatively, balloon 20 may be positioned with its distal edge closer to or farther from rim 17, depending on the desired application for the catheter.

First balloon 20 has an interior 22 that varies in volume when expanded and contracted. A second passage, shown as a second lumen 24, runs the length of first catheter 10 and communicates with interior 22 of the first balloon through intermediate apertures 26 that pass through tubing 12. A controlling fluid (not shown) flows within second lumen 24 and is controlled by an operator to expand/inflate and contract/deflate the first balloon. The first balloon functions as a flow-blocking mechanism to block the flow of blood or other fluid through a vessel while a surgical technique or process is being completed. As such, first balloon 20 is very compliant and inflates with a very slight change in pressure within second lumen 24. First balloon 20 preferably has an outer diameter of about 2-8 mm when fully inflated.

FIG. 2 shows catheter 10 inserted in a blood vessel V, typically an artery or vein, that is defined by a vessel wall W. Catheter 10 is threaded on a guidewire 28 that typically is inserted first into the blood vessel and maneuvered until the guidewire reaches a treatment site. Then the physician advances catheter 10 along guidewire 28 to place distal end 14 and aperture 16 of catheter 10 at the treatment site.

FIG. 3 shows catheter 10 with aperture 16 positioned at a treatment site. In this example, catheter 10 is used to kill or eliminate a desired tissue. For instance, in a case of idiopathic hypertrophic subaortic stenosis or if the septum of the heart is diseased, it may be necessary or desirable to kill the tissues comprising the septum of the heart. This may be accomplished by inserting a tissue-killing substance, such as alcohol, into the septum. First catheter 10 provides a way for such an alcohol infusion process to be performed without endangering the life of the patient. As shown in FIG. 3, the distance D between the distal end of balloon 20 and catheter distal aperture 16 may be selected for the particular application and may be smaller than that shown in FIGS. 1 and 2.

To perform this procedure, guide wire 28 is placed into the left anterior descending (LAD) coronary artery of the heart and into a septal branch S of the LAD artery (FIG. 9). First catheter 10 is guided along guide wire 28 until first balloon 20, in a contracted state, has entered septal branch S. The operator inflates first balloon 20 as previously described. An amount of alcohol A is released or delivered through first lumen 18 into septal branch S and is permitted to flow toward the septum (not shown), where the alcohol kills the tissue of the septum.

First balloon 20 serves as a blocking mechanism to prevent the flow of alcohol A out of the septal branch and into the LAD artery, where the alcohol would otherwise flow and destroy other tissues in the heart. By pressing against the interior wall W of septal branch S, first balloon 20 holds first catheter 10 in place while the alcohol is infused into the septal branch. Aperture 16 is located immediately adjacent first balloon 20, which enables an accurate delivery of alcohol relative to the first balloon. The operator completes the alcohol infusion process by deflating first balloon 20 and removing first catheter 10 and guide wire 28 from septal branch S and LAD artery.

It may sometimes be necessary to provide an electrical impulse to the heart after the alcohol infusion process is complete. This “pacing” of the heart may be accomplished by transmitting the electrical impulse through guide wire 28 prior to removing the guide wire from the septal branch or the LAD artery.

Another condition that catheter 10 may be used to treat are occlusions of blood vessels, including a chronic total occlusion which is a 100% blockages of a blood vessel that has been in existence for a significant time, typically clinically defined as 30 days or more. Catheter 10 may also be used in treating occlusions that have been in existence for a shorter period of time. Typically an occlusion becomes increasingly calcified the longer it remains in existence.

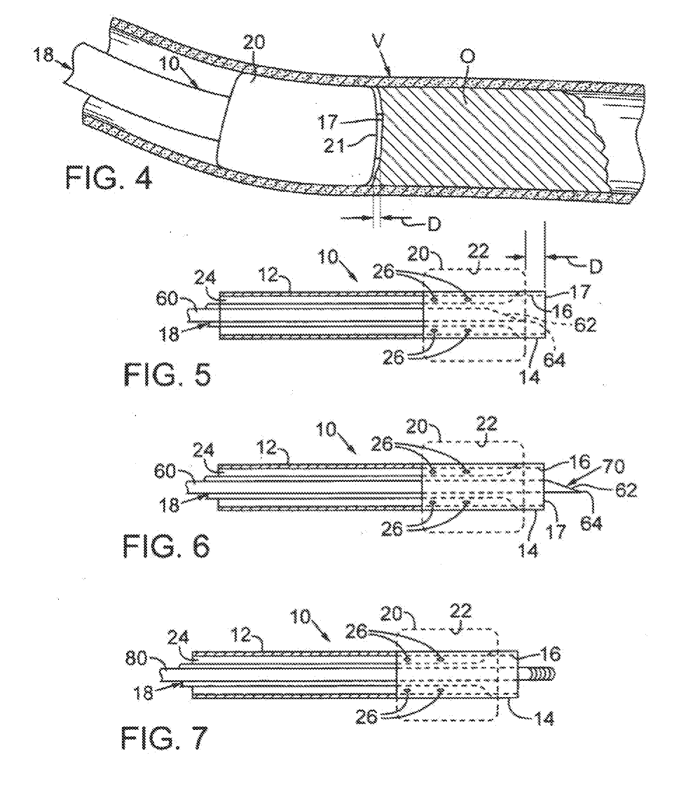

Catheter 10 is shown in FIG. 4 positioned at a treatment site for a total occlusion O. Preferably, treating such total occlusion with a liquid will involve confining the liquid to the tissue, plaque, and calcification of the total occlusion because, like the alcohol treatment described above, the liquid may be harmful to other tissue. Catheter 10 is preferably positioned with distal edge 17 butted up against occlusion O and balloon 20 is inflated. Balloon 20 holds the catheter in place and prevents the catheter from being inadvertently moved during a process.

Balloon 20 substantially seals off the wall W of vessel V proximal to distal end 14 of catheter 10. Balloon 20 also confines any liquid pumped through lumen 18 and out of aperture 16 to the tissue, plaque, and calcification of the occlusion. Some liquid may enter the area of vessel V between distal end 21 of balloon 20 and occlusion O. However, this area is limited by the separation D between distal end 21 of balloon 20 and distal edge 17 of catheter 10.

Two methods for treating a total occlusion are: (1) promoting the growth of collateral blood vessels and (2) dissolving the plaque and calcification to reopen the blood vessel. Either of these approaches may be carried out by the injection of a liquid through lumen 18 and out of aperture 16 of catheter 10 to infuse the occlusion.

Promotion of collaterals may be carried out by infusion with a vascular endothelial growth factor (VEGF), a fibroblast growth factor (FGF), or such other substances that tend to promote angiogenesis.

Dissolving the plaque and calcification may be carried out by infusion of a plasminogen activator, such as urokinase or thrombolytic plasminogen activator (tPA), or other thrombolytics or other solutions that will help in breaking up the occlusion. The liquid may be injected into the total occlusion and held there by maintaining inflation of the balloon to seal off the area outside the treatment site and protect other tissue from the liquid. The time period for holding the liquid in place may be selected for the expected resistance of the plaque and calcification to the desired dissolving. For example, the liquid may be flushed in and held in place, for a short period, such as 15-20 minutes, for an intermediate period of 2-3 hours, or a long period of 12-48 hours. Typically, after the liquid treatment is completed, the occlusion, or what is left of it, will be further treated by advancement of a wire through the occluded area. Alternatively, the liquid treatment and wire advancement may be performed together, i.e., advancing a wire while the liquid is still in place, or iteratively, i.e., advancing the wire partially through the occlusion, injecting more liquid, advancing the wire further, etc.

FIGS. 5, 6, 9, and 11 show a needle-tipped, hollow wire 60 for use with catheter 10. Wire 60 is typically inserted in lumen 18 of catheter 10. Wire 60 may be positioned, as shown in FIG. 5, so that a distal tip 62 of wire 60 does not extend beyond distal edge 17 of catheter 10. This is the preferred position for advancing catheter 10 in a blood vessel so that tip 62 does not cause trauma to the vessel. Distal tip 62 of wire 60 culminates in a sharp point 64. FIG. 6 shows wire 60 with distal tip 62 extending beyond distal end 14 of catheter 10, which is the typical position at the treatment site.

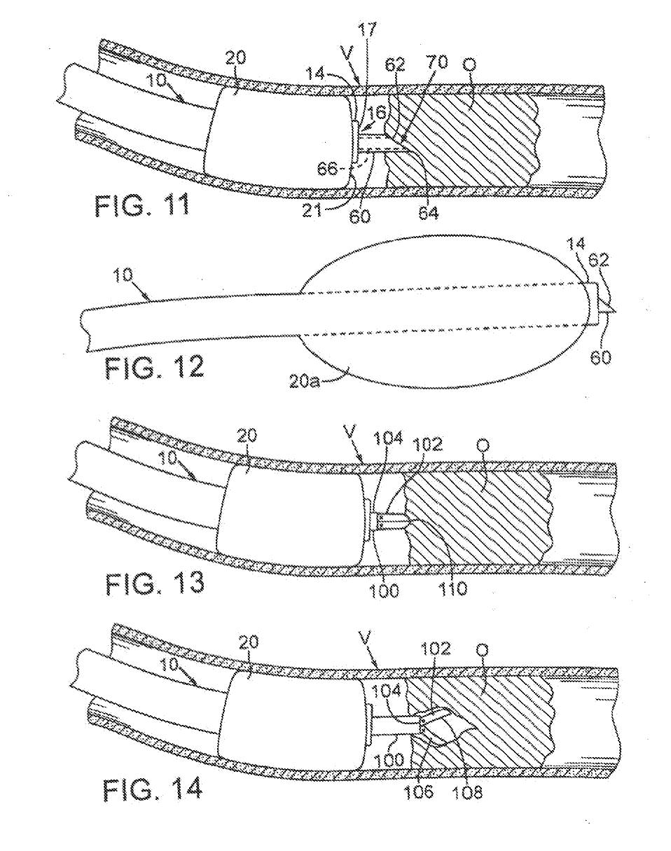

As best seen in FIGS. 9 and 11, wire 60 includes a lumen 66 extending from a proximal end 68 to a distal opening 70 at tip 62. Proximal end 68 of wire 60 may be coupled to an injection device, such as syringe 72 by a vacuum seal 74. A plunger 76 in syringe 72 may be depressed to inject a liquid through wire 60 and out distal tip 62, or plunger 76 may be withdrawn to create a vacuum to draw liquid into wire 60 at the distal tip.

FIG. 11 shows distal tip 62 of wire 60 extended beyond distal end 14 of catheter 10 and sharp point 64 inserted into occlusion O. Balloon 20 is inflated to seal off the treatment site. Liquid injected into occlusion O through wire 60 exits the wire at distal opening 70 and thus enters occlusion O at a depth within the plaque and calcification that is determined by the depth of insertion of distal tip 62 and the pressure with which the liquid is injected. The liquid is maintained in the treatment site by balloon 20 as described above. The position of distal tip 62 of wire 60 relative to distal edge 17 of catheter 10 may be selected and adjusted as desired by the physician, resulting in more or less area in the vessel between distal edge 21 of balloon 20 and occlusion O. In any case, the position of distal edge 21 of balloon 20 immediately adjacent aperture 16 and distal edge 17 of catheter 10 allows the physician to reduce the area as much as is desired.

As the occlusion is dissolved by infused liquid, wire 60 may be advanced through occlusion O. When tip 62 of wire 60 passes all the way through occlusion O, a stent and/or balloon catheter may be advanced through occlusion O and expanded to reopen the blood vessel.

FIGS. 7 and 10 show a wire 80 inserted through catheter 10. The position of wire 80 relative to catheter 10 is controlled by the physician and wire 80 may be withdrawn into lumen 18 of catheter 10 or extend beyond distal end 14 as shown in FIGS. 7 and 10. Wire 80 may be provided with a hot tip, or radio-frequency (RF) tip 82 which may be of the type described in U.S. Pat. No. 6,190,379, which is incorporated herein by reference. Wire 80 may also be provided with a lumen 84 (FIG. 10), as for wire 60, for the injection of liquids through wire 80 for infusion at a treatment site.

Wire 80 is shown in FIG. 10 at a treatment site for an occlusion O. Balloon 20 is inflated to prevent infused liquid or debris from use of the RF tip from leaving the treatment site. RF tip 82 is shown extending completely beyond distal end 14 of catheter 10, but the tip may be moved to any position relative to catheter 10 for a desired treatment. For example, wire 80 could be withdrawn so that less of tip 82 extends beyond distal end 14 to further confine infused liquid and/or debris.

As the occlusion is dissolved by infused liquid and/or ablated by the RF tip, wire 80 may be advanced through occlusion O. When tip 82 of wire 80 passes all the way through occlusion O, a stent and/or balloon catheter may be advanced through occlusion O and expanded to reopen the blood vessel.

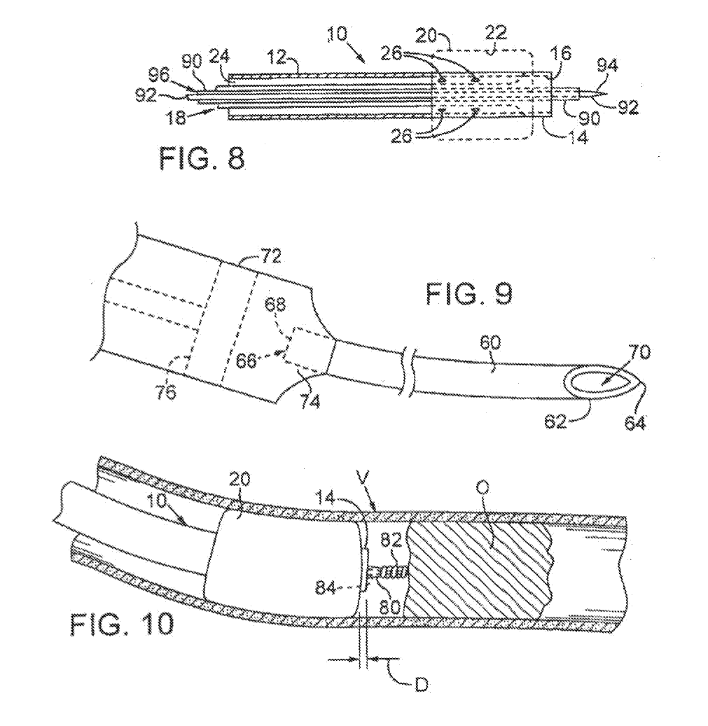

Another wire that may be used in catheter 10 is shown in FIG. 8, where a hollow wire 90 is inserted through lumen 18 of catheter 10. A needle wire 92 with a pointed tip 94 may be inserted through a lumen 96 of hollow wire 90. The relative positions of catheter 10, hollow wire 90, and needle wire 92 are under control of the physician, as for the wires described above. Thus, wires 90 and 92 may be used to infuse liquid and pierce through an occlusion as described above for wires 60 and 80.

Other wires may be used in conjunction with catheter 10 for the treatment of occlusions, for example, the Safe-Cross ® RF Crossing Wire made by Intraluminal Therapeutics, Inc. of Carlsbad, Calif. Alternatively, a laser wire could be used.

Another wire that can be used in catheter 10 is made by LuMend, Inc. of Redwood City, Calif. Such a wire 100 is shown in FIGS. 13 and 14 with a blunt micro-dissection tool 102 at a distal end 104 of wire 100. Tool 102 includes two jaws 106, 108, which when closed, as seen in FIG. 13, form a generally blunt tip 110 that engages occlusion O. As shown in FIG. 14, jaws 106 and 108 may be opened to push the plaque and calcification apart, allowing tool 102 and wire 100 to be advanced through the occlusion.

Catheter 10 or other wires may be used prior to operation of the Intraluminal, laser, or LuMend wires to infuse liquid to dissolve the plaque and calcification as described above. Typically, when the wire tip has been passed all the way through occlusion O, a stent and/or balloon catheter may be advanced through occlusion O and expanded to reopen the blood vessel.

Catheter 10 is typically used with a balloon that inflates to a substantially cylindrical shape, as shown, e.g., in FIG. 3. Alternatively, the balloon may be provided with another shape suitable for the desired application. For example, as shown in FIG. 12, catheter 10 may include a balloon 20a that inflates to a substantially oval shape. These and other balloons typically are disposed on catheter 10 as for balloon 20 and may be used in providing treatment as described above. FIG. 12 shows hollow, needle-tipped wire 60 inserted through catheter 10, with distal tip 62 extending beyond catheter distal end 14, but other wires may be used with balloon 20a.

While certain embodiments are disclosed herein, these specific embodiments as disclosed and illustrated herein are not to be considered in a limiting sense as numerous variations are possible. Applicant regards the disclosure to include all novel and non-obvious combinations and subcombinations of the various elements, features, functions and/or properties disclosed herein. No single feature, function, element or property of the disclosed embodiments is essential. The following claims define certain combinations and subcombinations which are regarded as novel and non-obvious. Other combinations and subcombinations of features, functions, elements and/or properties may be claimed through amendment of the present claims or presentation of new claims in this or a related application. Such claims are also regarded as included within the subject matter of the disclosure irrespective of whether they are broader, narrower, or equal in scope to the original claims.

I claim:

1. A catheterization system for treatment of a condition within a blood vessel, the system comprising:
a support catheter defining a proximal end, a distal end, a central lumen interconnecting the ends, and an aperture at the distal end, the catheter insertable into the blood vessel to a treatment area that is substantially distal of the distal end of the catheter, wherein the central lumen is configured to permit the advancement of a wire therethrough to the treatment area;
a flexible membrane mounted on an outside surface of the catheter and disposed immediately adjacent to the aperture at the distal end of the catheter while deflated, the flexible membrane including a proximal end and an opposite distal end, wherein the distal end of the catheter projects slightly beyond the distal end of the flexible membrane when the flexible membrane is fully inflated; wherein the flexible membrane, when inflated, has a substantially cylindrical shape and is positioned to minimize contact between the vessel and a treatment fluid and/or debris;
an inflation device selectively operable adjacent the proximal end of the catheter and coupled to the flexible membrane to be capable of expanding the membrane under pressure to create an atraumatic seal with a vessel wall and substantially close off the blood vessel; and
a fluid injection device selectively operable adjacent the proximal end of the catheter to inject a treatment fluid through the central lumen of the catheter and out of the aperture at the distal end of the catheter into the treatment area, wherein the treatment fluid is a tissue-killing fluid.

2. The catheterization system of claim 1, further wherein the aperture at the distal end of the catheter is defined by a distal edge of the catheter, and further wherein the distal end of the flexible membrane is no more than about 2-mm from the distal edge of the catheter.

3. The catheterization system of claim 1, further wherein the aperture at the distal end of the catheter is defined by a distal edge of the catheter, and further wherein the distal end of the flexible membrane is no more than about 1-mm from the distal edge of the catheter.

4. The catheterization system of claim 1, further wherein the aperture at the distal end of the catheter is defined by a distal edge of the catheter, and further wherein the distal end of the flexible membrane is no more than about 0.5 mm from the distal edge of the catheter.

5. The catheterization system of claim 1 wherein the catheter defines an outer diameter, and further wherein the aperture at the distal end of the catheter is defined by a distal edge of the catheter, and further wherein the distal end of the flexible membrane is a distance from the distal edge of the catheter that is no more than about one half of the outer diameter of the catheter.

6. The catheterization system of claim 1, further comprising an RF wire insertable through the central lumen of the catheter and selectively operable adjacent the proximal end of the catheter to apply RF energy to the treatment area.

7. The catheterization system of claim 1, further comprising a hollow wire insertable through the central lumen of the catheter and selectively operable adjacent the proximal end of the catheter to apply a fluid to the treatment area.

8. The catheterization system of claim 7, wherein the hollow wire further includes a distal needle tip.

9. The catheterization system of claim 1, further comprising a wire including a dissection tool adjacent a distal end, the wire insertable through the central lumen of the catheter and selectively operable adjacent the proximal end of the catheter to dissect plaque in the treatment area.

10. The catheterization system of claim 1, further comprising a wire including a laser delivery device adjacent a distal end, the wire insertable through the central lumen of the catheter and selectively operable adjacent the proximal end of the catheter to apply laser energy to the treatment area.
